(19) United States
(12) Patent Application Publication
(10) Pub. No.: US 2026/0018172 A1
(43) Pub. Date: Jan. 15, 2026

(54) INFORMATION CONVERSION SYSTEM, INFORMATION PROCESSING METHOD, AND STORAGE MEDIUM

(71) Applicant: CANON KABUSHIKI KAISHA, Tokyo (JP)

(72) Inventors: JUNYA TANAKA, Kanagawa (JP); CHIHIRO NAGURA, Tochigi (JP); TAIHEI MUKAIDE, Kanagawa (JP)

(21) Appl. No.: 19/335,369

(22) Filed: Sep. 22, 2025

Related U.S. Application Data
(63) Continuation of application No. PCT/JP2024/011841, filed on Mar. 26, 2024.

(30) Foreign Application Priority Data
Mar. 27, 2023 (JP) 2023-050405

(51) Int. Cl. G10L 15/25; A61B 5/00; G06F 3/0346

(52) U.S. Cl. CPC G10L 15/25; A61B 5/4803; A61B 5/721; G06F 3/0346; A61B 2562/0219

(57) ABSTRACT

Provided is an information conversion system capable of reducing body movement information from biometric information and converting the biometric information into character information or voice information. The information conversion system according to the present disclosure includes biometric information detection units 101 and 102 each configured to detect biometric information from one or more parts of a user; a body movement information detection unit 103 configured to detect the body movement information of the user, a body movement reduction processing unit 110 configured to reduce the body movement information from the biometric information; and a conversion unit 106 configured to convert the biometric information from which the body movement information is reduced into character information or voice information.

### CROSS-REFERENCE TO RELATED APPLICATIONS

This application is a Continuation of International Patent Application No. PCT/JP2024/011841, filed Mar. 26, 2024, which claims the benefit of Japanese Patent Application No. 2023-050405, filed Mar. 27, 2023, both of which are hereby incorporated by reference herein in their entirety.

### BACKGROUND

### Field of the Technology

The present disclosure relates to an information conversion system that converts biometric information into character information or voice information, an information processing method, and a storage medium.

### Description of the Related Art

In recent years, speech content has been recognized using user's voice information. On the other hand, voice biometric information has been acquired instead of voice information, and a facial expression, character information, or the like of a user has been recognized from the biometric information and output (for example, Information Processing Society of Japan, Interaction 2020, “Derma: Silent Speech Interaction via Cutaneous Motion Measurement.”).

In Information Processing Society of Japan, Interaction 2020, “Derma: Silent Speech Interaction via Cutaneous Motion Measurement.”, identification of a phrase is performed from biometric information regarding skin movement, by moving a mouth without vocalization for a phrase preliminarily set, with use of an acceleration sensor and an angular velocity sensor.

In Information Processing Society of Japan, Interaction 2020, “Derma: Silent Speech Interaction via Cutaneous Motion Measurement.”, however, data is acquired with a focus on the skin movement, but no consideration is given to user's body movement. If the user's body movement occurs while user's biometric information is being detected, body movement information (body movement component) is added to the biometric information.

However, an issue to be solved by embodiments described in the present specification and drawings is not limited to the above-mentioned issue. An issue corresponding to each effect achieved by each configuration described in the embodiments to be described below may be positioned as another issue.

### SUMMARY

The present disclosure is directed to reducing body movement information from biometric information and converting the biometric information into character information or voice information.

To achieve the purpose of the present disclosure, an information conversion system includes a biometric information detection unit configured to detect biometric information from one or more parts of a user, a body movement information detection unit configured to detect body movement information of the user, a body movement reduction processing unit configured to reduce the body movement information from the biometric information, and a conversion unit configured to convert the biometric information from which the body movement information is reduced into character information or voice information.

Further, an information conversion method includes steps of detecting biometric information from one or more parts of a user, detecting body movement information of the user, reducing the body movement information from the biometric information, and converting the biometric information from which the body movement information is reduced into character information or voice information.

Features of the present disclosure will become apparent from the following description of embodiments with reference to the attached drawings.

### BRIEF DESCRIPTION OF THE DRAWINGS

FIG. 1 is a diagram illustrating a configuration of an information conversion system according to the present disclosure.

FIG. 2 includes charts illustrating processing of a conversion unit in the information conversion system according to the present disclosure.

FIG. 3 is a schematic view illustrating an example of a detection device according to the present disclosure.

FIG. 4 is a flowchart illustrating an operation of the information conversion system according to the present disclosure.

FIG. 5 is a schematic view illustrating an example of a detection device according to the present disclosure.

FIG. 6 includes schematic views illustrating an example of a detection device according to the present disclosure.

### DESCRIPTION OF THE EMBODIMENTS

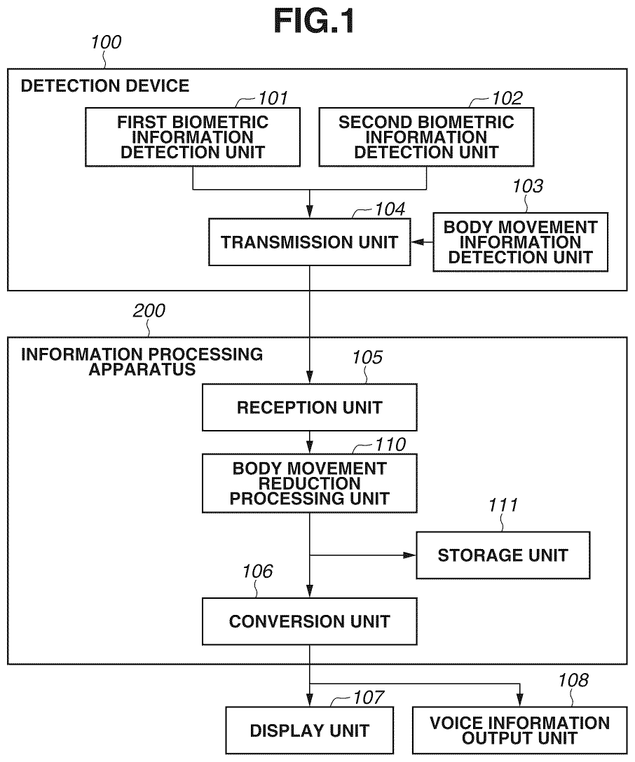

Embodiments of the present disclosure will be described in detail below. FIG. 1 schematically illustrates an information conversion system according to the present disclosure.

The information conversion system is composed mainly of a detection device 100 and an information processing apparatus 200. The detection device 100 includes a first biometric information detection unit 101, a second biometric information detection unit 102, a body movement information detection unit 103, and a transmission unit 104. The first biometric information detection unit 101 and the second biometric information detection unit 102 each detect biometric information from one or more body parts of a user. The body movement information detection unit 103 detects body movement information about the user. The transmission unit 104 transmits the biometric information detected by the first biometric information detection unit 101, the biometric information detected by the second biometric information detection unit 102, and the body movement information detected by the body movement information detection unit 103 to the information processing apparatus 200.

The first biometric information detection unit 101 and the second biometric information detection unit 102 detect biometric information at respective different positions. Each of the first biometric information detection unit 101 and the second biometric information detection unit 102 is, for example, an acceleration sensor that detects the movement of the user's mouth and tongue. Alternatively, each of the first biometric information detection unit 101 and the second biometric information detection unit 102 may be an acceleration sensor and an angular velocity sensor that detect the movement of the user's mouth and tongue.

The acceleration sensor is a sensor that detects acceleration and outputs data or a signal corresponding to the detected acceleration. The angular velocity sensor (gyro sensor) is a sensor that detects angular velocity and outputs data or a signal corresponding to the detected angular velocity. The acceleration sensor and the angular velocity sensor in each of the first biometric information detection unit 101 and the second biometric information detection unit 102 may be integrated.

Each of the first biometric information detection unit 101 and the second biometric information detection unit 102 is installed at a position where the movement of the user's mouth and tongue is detectable. Each of the first biometric information detection unit 101 and the second biometric information detection unit 102 is installed in the vicinity of the user's mouth. Specifically, each of the first biometric information detection unit 101 and the second biometric information detection unit 102 is installed in the user's lower jaw portion, cheek, throat, or the like. Each of the first biometric information detection unit 101 and the second biometric information detection unit 102 may be installed in a body part other than the above-mentioned body parts as long as the biometric information regarding the movement of the mouth and tongue is detectable at the position.

The body movement information detection unit 103 is, for example, an acceleration sensor that detects user's body movement information. The body movement information detection unit 103 may be an acceleration sensor and an angular velocity sensor that detect the user's body movement information. The acceleration sensor and the angular velocity sensor in the body movement information detection unit 103 may be integrated.

A sensor used in the body movement information detection unit 103 and a sensor used in each of the first biometric information detection unit 101 and the second biometric information detection unit 102 are of an identical type, and have equivalent measurement performance such as a measurement range, a response frequency, or a sampling period. For example, the acceleration sensor used in the body movement information detection unit 103 and the acceleration sensor used in each of the first biometric information detection unit 101 and the second biometric information detection unit 102 are of an identical type.

Types of the acceleration sensor include a piezoelectric type, a piezoresistive type, and a static capacitance type. Specifically, if the acceleration sensor used in the body movement information detection unit 103 is of the piezoelectric type, the acceleration sensor used in each of the first biometric information detection unit 101 and the second biometric information detection unit 102 is of the piezoelectric type. If the acceleration sensor used in the body movement information detection unit 103 is of the static capacitance type, the acceleration sensor used in each of the first biometric information detection unit 101 and the second biometric information detection unit 102 is of the static capacitance type.

The acceleration sensor used in the body movement information detection unit 103 and the acceleration sensor used in each of the first biometric information detection unit 101 and the second biometric information detection unit 102 have equivalent measurement performance such as a measurement range, a response frequency, or a sampling period.

Further, the angular velocity sensor used in the body movement information detection unit 103 and the angular velocity sensor used in each of the first biometric information detection unit 101 and the second biometric information detection unit 102 are of an identical type.

Types of the angular velocity sensor include a piezoelectric type and a static capacitance type. For example, if the angular velocity sensor used in the body movement information detection unit 103 is of the piezoelectric type, the angular velocity sensor used in each of the first biometric information detection unit 101 and the second biometric information detection unit 102 is of the piezoelectric type. If the angular velocity sensor used in the body movement information detection unit 103 is of the static capacitance type, the angular velocity sensor used in each of the first biometric information detection unit 101 and the second biometric information detection unit 102 is of the static capacitance type.

The angular velocity sensor used in the body movement information detection unit 103 and the angular velocity sensor used in each of the first biometric information detection unit 101 and the second biometric information detection unit 102 have equivalent measurement performance such as a measurement range, a response frequency, or a sampling period.

The body movement information detection unit 103 is installed at a position that is not influenced by the movement of the user's mouth and tongue, in other words, at a position where the movement of the user's mouth and tongue is not detectable. Specifically, the body movement information detection unit 103 is installed in the vicinity of the user's neck, ear, or the like. The body movement information detection unit 103 may be installed in a body part other than the above-mentioned body parts as long as the body movement information detection unit 103 is not influenced by the movement of the user's mouth at the position.

While FIG. 1 illustrates a mode in which the detection device 100 is composed of two biometric information detection units, the detection device 100 may be a device including three or more biometric information detection units. The detection device 100 may also include a mechanism that acquires voice information in addition to the biometric information. Further, the detection device 100 can be restated as an acquisition unit that acquires biometric information.

Each of the first biometric information detection unit 101 and the second biometric information detection unit 102 is composed of a sensor that detects biometric information regarding the movement of the user's mouth and tongue. Each of the first biometric information detection unit 101 and the second biometric information detection unit 102 may include, in addition to the acceleration sensor and the angular velocity sensor, a geomagnetic sensor, a electromyographic sensor, an ultrasonic sensor, a tactile sensor, an optical sensor, and a pressure sensor. Specifically, each of the first biometric information detection unit 101 and the second biometric information detection unit 102 may be the acceleration sensor, or the acceleration sensor and the angular velocity sensor in which the geomagnetic sensor, the electromyographic sensor, the pressure sensor, or the like is incorporated. The first biometric information detection unit 101 and the second biometric information detection unit 102 are capable of acquiring information regarding skin movement and muscle movement in different body parts of the user.

In a case where each of the first biometric information detection unit 101 and the second biometric information detection unit 102 is composed of two types of sensors, a measurement timing and a sampling frequency may be different between the sensors.

The information processing apparatus 200 includes a reception unit 105, a body movement reduction processing unit 110, a conversion unit 106, and a storage unit 111. The reception unit 105 receives the biometric information transmitted from the detection device 100. The body movement reduction processing unit 110 reduces the body movement information (body movement component) in the biometric information. The conversion unit 106 converts the biometric information, in which the body movement information has been reduced, into character information or voice information. The storage unit 111 stores a trained model to be used when the biometric information, in which the body movement information has been reduced, is converted into the character information or the voice information in the conversion unit 106.

The information processing apparatus 200 refers to a smartphone, a personal computer (PC), or a tablet PC, but is not limited thereto. In a case where the information processing apparatus 200 is the PC, for example, the character information obtained by conversion by the conversion unit 106 is transmitted to a display unit 107, such as a display.

The display unit 107 displays the character information obtained by conversion of the biometric information. The information processing apparatus 200 may include a display control unit (not illustrated) that controls a display mode in the display unit 107.

The body movement reduction processing unit 110 reduces the body movement information from the biometric information received by the reception unit 105. Specifically, the body movement reduction processing unit 110 subtracts a waveform based on the body movement information detected by the body movement information detection unit 103 from a waveform based on the biometric information detected by the first biometric information detection unit 101. Further, the body movement reduction processing unit 110 subtracts the waveform based on the body movement information detected by the body movement information detection unit 103 from a waveform based on the biometric information detected by the second biometric information detection unit 102. Then, a waveform obtained by subtracting the waveform based on the body movement information from the waveform based on the biometric information, i.e., the biometric information from which the body movement information is reduced, is input to the conversion unit 106. In a case where there is a plurality of waveforms each obtained by subtracting the waveform based on the body movement information from the waveform based on the biometric information, the plurality of waveforms is input to the conversion unit 106.

The conversion unit 106 converts the waveform obtained by subtracting the waveform based on the body movement information from the waveform based on the biometric information into the character information or the voice information. In a case where there is the plurality of waveforms each obtained by subtracting the waveform based on the body movement information from the waveform based on the biometric information, the conversion unit 106 uses the plurality of waveforms each obtained by subtracting the waveform based on the body movement information from the waveform based on the biometric information to generate the character information or the voice information.

Specifically, the conversion unit 106 uses a first waveform obtained by subtracting the waveform based on the body movement information detected by the body movement information detection unit 103 from the waveform based on the biometric information detected by the first biometric information detection unit 101 and a second waveform obtained by subtracting the waveform based on the body movement information detected by the body movement information detection unit 103 from the waveform based on the biometric information detected by the second biometric information detection unit 102 to generate the character information or the voice information.

In short, the conversion unit 106 converts a plurality of pieces of biometric information from which body movement information is reduced into one piece of character information or one piece of voice information.

In this manner, by converting biometric information from which an acceleration component and an angular velocity component associated with a body movement different from a mouth movement are reduced into character information or voice information, it is possible to generate the character information or the voice information in a form in which recognition accuracy is increased.

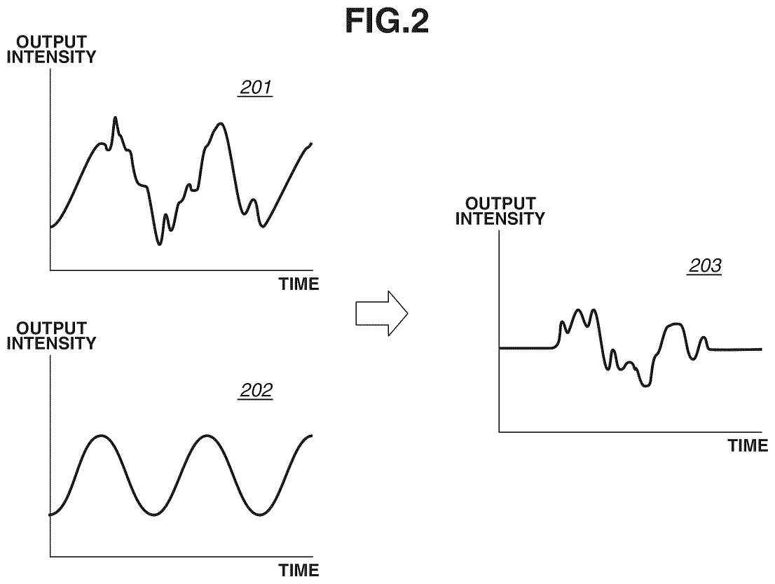

FIG. 2 illustrates waveforms based on the biometric information and the body movement information, in which the horizontal axis represents time and the vertical axis represents output intensity. FIG. 2 illustrates one axis of acceleration or angular velocity for the sake of simplification of explanation. However, the waveforms may be waveforms corresponding to three axes corresponding to three degrees of freedom of acceleration (an X-axis direction, a Y-axis direction, and a Z-axis direction), or six axes in total including the three axes corresponding to the three degrees of freedom of acceleration and three axes corresponding to three degrees of freedom of angular velocity (rotation around an X-axis, a Y-axis, and a Z-axis).

A waveform 201 is a waveform based on the biometric information detected by the first biometric information detection unit 101 when the user's body movement occurs, for example, when the body sways laterally. The waveform 201 may be the biometric information detected by the second biometric information detection unit 102.

A waveform 202 is a waveform indicating occurrence of the user's body movement, i.e., lateral swaying of the body, in the body movement information detected by the body movement information detection unit 103. The body movement reduction processing unit 110 subtracts the waveform 202 indicating the user's body movement information from the waveform 201 to extract a waveform 203.

In FIG. 2, the waveform 203 at the time of speech is obtained by subtracting the waveform 202, which is based on the lateral swaying of the body and is output by the body movement information detection unit 103, from the waveform 201, which is based on the speech (the mouth movement) at the time of lateral swaying of the body (the body movement). In other words, the waveform 203 indicates the biometric information from which the body movement information (body movement component) is reduced. In the case of executing on the three axes with regard to acceleration, the biometric information from which the body movement is reduced on the three axes is similarly output. In the case of executing on the six axes with regard to acceleration and angular velocity, the biometric information from which the body movement is reduced on the six axes is similarly output.

Further, in the acceleration sensor and the angular velocity sensor, it is necessary to match the X-axis direction, the Y-axis direction, and the Z-axis direction in each part. Each of the acceleration sensor and the angular velocity sensor may be a geomagnetic sensor that can easily match the X-axis direction, the Y-axis direction, and the Z-axis direction.

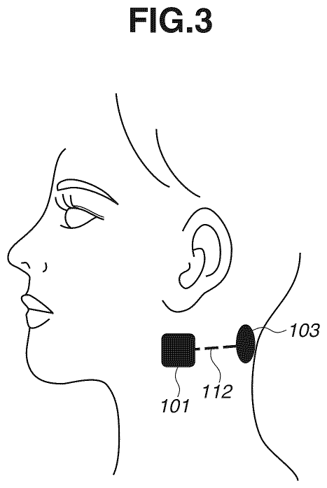

Then, as an example of a method for further increasing the accuracy, the first biometric information detection unit 101 and the body movement information detection unit 103 are installed in a relationship as illustrated in FIG. 3. Although not illustrated, the second biometric information detection unit 102 may be installed at an opposite position (below the right ear) to the position of the first biometric information detection unit 101 (below the left ear).

A relative position vector of the body movement information detection unit 103 relative to the first biometric information detection unit 101 is a vector 112. Here, the acceleration detected by the body movement information detection unit 103, the angular velocity detected by the body movement information detection unit 103, the acceleration detected by the first biometric information detection unit 101, the angular velocity detected by the first biometric information detection unit 101, the acceleration detected by the second biometric information detection unit 102, and the angular velocity detected by the second biometric information detection unit 102 are acquired. By calculating a difference in acceleration and a difference in angular velocity, it is possible to reduce the body movement information from the biometric information. For example, the body movement reduction processing unit 110 is capable of reducing the body movement information from the biometric information by subtracting the acceleration detected by the body movement information detection unit 103 from the acceleration detected by the first biometric information detection unit 101 or subtracting the angular velocity detected by the body movement information detection unit 103 from the angular velocity detected by the first biometric information detection unit 101. The body movement reduction processing unit 110 is capable of reducing the body movement information from the biometric information by subtracting the acceleration detected by the body movement information detection unit 103 from the acceleration detected by the second biometric information detection unit 102 or subtracting the angular velocity detected by the body movement information detection unit 103 from the angular velocity detected by the second biometric information detection unit 102.

Before information is input to the conversion unit 106, the acceleration may be subjected to, not only mere subtraction, but also to pre-processing such as noise removal.

Description is given below of a specific example of processing of reducing the body movement in the body movement reduction processing unit 110 in a state where the X-axis direction, the Y-axis direction, and the Z-axis direction are matched. In a stationary state, the body movement reduction processing unit 110 acquires three-axis accelerations ax, ay, and az from a three-axis acceleration sensor. The body movement reduction processing unit 110 uses ax, ay, and az to calculate a roll angle r and a pitch angle p in a sensor coordinate system with a gravitational acceleration direction serving as a reference by the following computation.

$$r = {tan}^{- 1} ( \frac{{a}_{y}}{{a}_{z}} ) \qquad [ Equation 1 ]$$

$$p = - {tan}^{- 1} ( \frac{{a}_{x}}{\sqrt{{a}_{y}^{2} + {a}_{z}^{2}}} ) \qquad [ Equation 2 ]$$

Subsequently, the body movement reduction processing unit 110 acquires magnetic intensities mx, my, and mz along three orthogonal axes by a three-axis geomagnetic sensor. The body movement reduction processing unit 110 uses mx, my, and mz to calculate a yaw angle y in the sensor coordinate system with a geomagnetic direction serving as a reference by the following computation.

$$y = - {tan}^{- 1} ( \frac{{m}_{y} cos r - {m}_{z} sin r}{{m}_{x} cos p + {m}_{y} sin p sin r + {m}_{z} sin p cos r} ) \qquad [ Equation 3 ]$$

The body movement reduction processing unit 110 uses the roll angle r, the pitch angle p, and the yaw angle y that have been obtained above to calculate a rotation matrix R by the following computation.

$$R = [ \begin{matrix}sin p cos y & - cos p sin y & sin p \\ cos p sin y + cos y sin r sin p & cos r cos y - sin r sin p sin y & - cos p sin r \\ sin r sin y - cos r cos y sin p & cos y sin r + cos r sin p sin y & cos r cos p\end{matrix} ] \qquad [ Equation 4 ]$$

A rotation matrix calculated with the sensor of the body movement information detection unit 103 is defined as R0. A rotation matrix calculated with the sensor in the first biometric information detection unit 101 is defined as R1. A rotation matrix R01 that converts a sensor coordinate system of the body movement information detection unit 103 into a sensor coordinate system of the first biometric information detection unit 101 is determined as follows.

$$R 01 = R 1 R 0 T$$

In a recognition operation, the body movement reduction processing unit 110 performs rotation conversion on outputs ax0, ay0, and az0 of the three-axis acceleration sensor in the body movement information detection unit 103 with use of the rotation matrix R01, and thereby obtains outputs ax0′, ay0′, and az0′ of the acceleration sensor in consistency with the sensor coordinate system of the first biometric information detection unit 101.

$$[ \begin{matrix}{a}_{x 0}^{′} \\ {a}_{y 0}^{′} \\ {a}_{z 0}^{′}\end{matrix} ] = {R}_{01} [ \begin{matrix}{a}_{x 0} \\ {a}_{y 0} \\ {a}_{z 0}\end{matrix} ] \qquad [ Equation 5 ]$$

Similarly, the body movement reduction processing unit 110 performs rotation conversion on outputs ωx0, ωy0, and ωz0 of the three-axis angular velocity in the body movement information detection unit 103 with use of the rotation matrix R01, and thereby obtains outputs ωx0′, ωy0′, and ωz0′ of the angular velocity sensor in consistency with the sensor coordinate system of the first biometric information detection unit 101.

$$[ \begin{matrix}{ω}_{x 0}^{′} \\ {ω}_{y 0}^{′} \\ {ω}_{z 0}^{′}\end{matrix} ] = {R}_{01} [ \begin{matrix}{ω}_{x 0} \\ {ω}_{y 0} \\ {ω}_{z 0}\end{matrix} ] \qquad [ Equation 6 ]$$

The body movement reduction processing unit 110 obtains outputs ωxc, ωyc, and ωzc of the angular velocity sensor in the first biometric information detection unit 101 by the following computation. In the outputs ωxc, ωyc, and ωzc, the body movement is corrected with use of ωx0′, ωy0′, and ωz0′ obtained as described above and outputs ωx1, ωy1, and ωz1 of the angular velocity sensor in the first biometric information detection unit 101.

$$[ \begin{matrix}{ω}_{xc} \\ {ω}_{yc} \\ {ω}_{zc}\end{matrix} ] = [ \begin{matrix}{ω}_{x 1} \\ {ω}_{y x} \\ {ω}_{z 1}\end{matrix} ] - [ \begin{matrix}{ω}_{x 0}^{′} \\ {ω}_{y 0}^{′} \\ {ω}_{z 0}^{′}\end{matrix} ] \qquad [ Equation 7 ]$$

Further, the body movement reduction processing unit 110 obtains outputs axc, ayc, and azc of the acceleration sensor in the first biometric information detection unit 101 by the following computation. In the outputs axc, ayc, and azc, the body movement is corrected with use of ωx0′, ωy0′, and ωz0′ and outputs ax1, ay1, and az1 of the acceleration sensor in the first biometric information detection unit 101.

$$[ \begin{matrix}{a}_{xc} \\ {a}_{yc} \\ {a}_{zc}\end{matrix} ] = [ \begin{matrix}{a}_{x 1} \\ {a}_{y x} \\ {a}_{z 1}\end{matrix} ] - l \times [ \begin{matrix}{\dot{ω}}_{x 0}^{′} \\ {\dot{ω}}_{y 0}^{′} \\ {\dot{ω}}_{z 0}^{′}\end{matrix} ] \qquad [ Equation 8 ]$$

Here, 1 represents the relative position vector 112 of a sensor position of the body movement information detection unit 103 with respect to a sensor position of the first biometric information detection unit 101 as viewed in the sensor coordinate system of the first biometric information detection unit 101.

If the positional relationship between the first biometric information detection unit 101 and the body movement information detection unit 103 is uniquely determined, the relative position vector 1 may be determined based on a design value or may be measured and determined at the time of shipment. Furthermore, the positional relationship is established without the geomagnetic sensor.

Here, the description has been given of generation of the outputs axc, ayc, azc of the acceleration sensor, in which the body movement is corrected, in the first biometric information detection unit 101 for the sake of simplification of explanation. However, it is also possible to similarly generate outputs bxc, byc, bzc of the acceleration sensor, in which the body movement is corrected, in the second biometric information detection unit 102.

The conversion unit 106 converts the biometric information from which the body movement information is reduced into the character information or the voice information. The conversion unit 106 is capable of further converting the character information obtained by conversion into the voice information. Further, the conversion unit 106 is also capable of directly converting the biometric information into the voice information. The voice information obtained by conversion by the conversion unit 106 is transmitted to an voice information output unit 108. The voice information output unit 108 is a speaker and is capable of reproducing the voice information.

When converting the biometric information that is detected by the first biometric information detection unit 101 and from which the body movement information is reduced and the biometric information that is detected by the second biometric information detection unit 102 and from which the body movement information is reduced into the character information or the voice information, the conversion unit 106 uses a predetermined conversion method to convert the biometric information into the character information or the voice information.

For a conversion algorithm in the conversion method, a trained model using an architecture composed of a neural network is used. The information processing apparatus 200 includes the storage unit 111 that stores the trained model. The conversion unit 106 has a function of performing inference using the trained model.

The trained model is a model generated with use of a convolutional neural network (CNN) or a recurrent neural network (RNN) that performs deep learning. The trained model may be a model derived from the CNN or the RNN.

The information processing apparatus 200, for example, performs training by associating the biometric information detected by the first biometric information detection unit 101 and the biometric information detected by the second biometric information detection unit 102 with the character information or the voice information to generate the trained model to be used in the conversion method.

Specifically, the information processing apparatus 200 preliminarily acquires a plurality of datasets in which each of the biometric information detected by the first biometric information detection unit 101 and the biometric information detected by the second biometric information detection unit 102 is associated with the character information, the voice information (for example, a, i, u, e, o), or sounds of these pieces of information. Each of the first biometric information detection unit 101 and the second biometric information detection unit 102 is, for example, installed in a part in contact with the user's skin (contact surface). It is possible to measure biometric information regarding the user's movement from information from the acceleration sensor and the angular velocity sensor that are installed on the user.

Although not illustrated in FIG. 1, a training unit is configured in the information processing apparatus 200. Alternatively, the training unit may be configured on a cloud.

The training unit preliminarily acquires a plurality of datasets in which each of the biometric information detected by the first biometric information detection unit 101 and the biometric information detected by the second biometric information detection unit 102 is associated with the character information or the voice information. The training unit performs training by associating the biometric information with the character information or the voice information with use of a correspondence relationship between the biometric information and the character information or the voice information in each of the plurality of datasets as training data to generate the trained model. Note that the body movement information (body movement component) is not included in each of the biometric information detected by the first biometric information detection unit 101 and the biometric information detected by the second biometric information detection unit 102 to be used in training by the training unit (generation of the trained model).

In this manner, the conversion unit 106 uses the trained model that has been trained by associating the biometric information and the character information with each other to perform inference on the biometric information from which the body movement information (body movement component) is reduced, and is thereby capable of outputting the character information or the voice information.

The training unit is also capable of fine-tuning the original trained model by associating the biometric information with the character information or the voice information with use of the correspondence relationship between the biometric information and the character information or the voice information in the plurality of datasets as training data.

Alternatively, the conversion unit 106 is also capable of using different conversion algorithms (trained models) in accordance with the user's silent speech period and vocalized speech period. The silent speech period is a period during which the user does not produce voice. The vocalized speech period is a period during which the user produces voice.

The training unit is also capable of generating respective trained models by distinguishing between a body movement period and a rest period. Specifically, the training unit performs training by distinguishing between the body movement period and the rest period and associating the biometric information with the character information or the voice information with use of the correspondence relationship between the biometric information and the character information or the voice information in the plurality of datasets as training data to generate the respective trained models. The conversion unit 106 applies the trained model for the body movement period and the trained model for the rest period depending on whether the user is in the body movement period or the rest period. The conversion unit 106 is capable of performing inference on newly input biometric information during the body movement period or newly input biometric information during the rest period, and outputting character information or voice information.

Further, the training unit generates respective trained models by distinguishing between the silent speech period and the vocalized speech period. Specifically, the training unit performs training by distinguishing between the silent speech period and the vocalized speech period and associating the biometric information with the character information or the voice information with use of the correspondence relationship between the biometric information and the character information or the voice information in the plurality of datasets as training data to generate the respective trained models. The conversion unit 106 applies the trained model for the silent speech period and the trained model for the vocalized speech period depending on whether the user is in the silent speech period or the vocalized speech period. The conversion unit 106 is capable of performing inference on newly input biometric information in the silent speech period or newly input biometric information in the vocalized speech period, and outputting the character information or the voice information.

Alternatively, the information processing apparatus 200 may be configured on a cloud. The transmission unit 104 in the detection device 100 transmits the biometric information detected by the first biometric information detection unit 101 and the biometric information detected by the second biometric information detection unit 102 to the cloud. On the cloud, the biometric information is converted into the character information or the voice information, and the character information obtained by conversion is transmitted to the display unit 107. The display unit 107 displays the character information. Further, the information processing apparatus 200 may convert the character information obtained by conversion into the voice information and transmit the voice information to the voice information output unit 108. The information processing apparatus 200 may also directly convert the biometric information into the voice information and transmit the voice information to the voice information output unit 108.

Communication between the detection device 100 and the information processing apparatus 200 may be wired communication or wireless communication. In a case where the communication between the detection device 100 and the information processing apparatus 200 is implemented as wired communication, the transmission unit 104 in the detection device 100 and the reception unit 105 in the information processing apparatus 200 are connected by a wire such as a universal serial bus (USB) cable.

In a case where the communication between the detection device 100 and the information processing apparatus 200 is implemented as wireless communication, the transmission unit 104 in the detection device 100 and the reception unit 105 in the information processing apparatus 200 are wirelessly connected by wireless LAN communication such as Wireless Fidelity (Wi-Fi) or short-range wireless communication such as Bluetooth (registered trademark).

If the information processing apparatus 200 is a smartphone or a tablet PC, the display unit 107 is a display. The voice information output unit 108 is a speaker installed in the smartphone or the tablet PC, or earphones connected to the smartphone or the tablet PC.

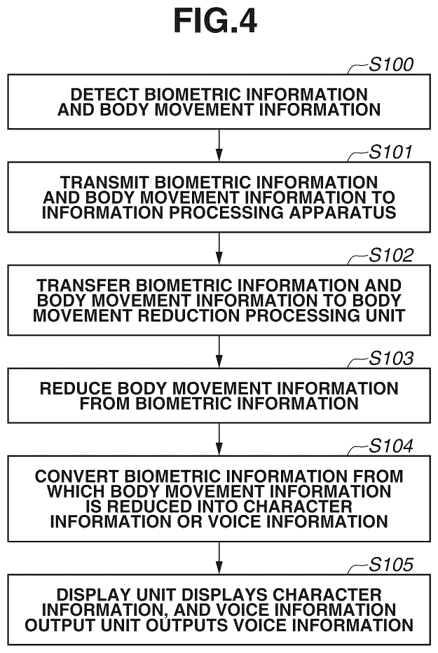

FIG. 4 is a flowchart illustrating operation performed by the information conversion system according to the present disclosure.

The user wears the detection device 100 at a position where the biometric information is detectable by the first biometric information detection unit 101 and the second biometric information detection unit 102. The user wears the detection device 100 on the head, on the neck portion, in the vicinity of the head or the neck portion, or the like. The first biometric information detection unit 101 and the second biometric information detection unit 102 each detect biometric information including at least one of acceleration, angular velocity, a magnetic signal, a myoelectric potential signal, pressure information, and the like. The body movement information detection unit 103 detects the user's body movement information (S100).

The transmission unit 104 transmits the biometric information and the body movement information to the information processing apparatus 200. Supplementary information regarding time information (timestamp information) is added to the biometric information and the body movement information. The supplementary information associates the biometric information and the body movement information with each other. Measurement times (timestamps) of the biometric information and the body movement information are matched. The transmission unit 104 is capable of transmitting the biometric information and the body movement information together with the supplementary information regarding the time information (timestamp information) to the information processing apparatus 200 (S101).

Each of the biometric information and the body movement information detected by the detection device 100 is received by the reception unit 105 of the information processing apparatus 200. The reception unit 105 transfers the biometric information and the body movement information to the body movement reduction processing unit 110 (S102).

The body movement reduction processing unit 110 reduces the body movement information from the biometric information. Specifically, the body movement reduction processing unit 110 subtracts the body movement information detected by the body movement information detection unit 103 from the biometric information detected by the first biometric information detection unit 101. Further, the body movement reduction processing unit 110 subtracts the body movement information detected by the body movement information detection unit 103 from the biometric information detected by the second biometric information detection unit 102. The biometric information from which the body movement information is subtracted and in which the body movement information is reduced is input to the conversion unit 106 (S103).

The conversion unit 106 converts the biometric information from which the body movement information is reduced into the character information or the voice information. Further, the conversion unit 106 may convert the biometric information from which the body movement information is reduced into the voice information, or may convert the biometric information into the character information and then perform voice synthesis to convert the character information into the voice information. The conversion unit 106 outputs the character information obtained by converting the biometric information from which the body movement information is reduced (S104).

The display unit 107 displays the character information obtained by conversion by the conversion unit 106. The voice information output unit 108 outputs the voice information obtained by further converting the character information obtained by conversion by the conversion unit 106 (S105).

The character information obtained by conversion by the conversion unit 106 can be stored in a storage unit of the information processing apparatus 200. The character information can be transferred via a network, and displayed on an external terminal.

As described above, the information conversion system according to the present disclosure includes the biometric information detection units 101 and 102 that detect the biometric information from one or more parts of the user, the body movement information detection unit 103 that detects the user's body movement information, the body movement reduction processing unit 110 that reduces the body movement information from the biometric information, and the conversion unit 106 that converts the biometric information from which the body movement information is reduced into the character information or the voice information. Further, an information conversion method includes a step of detecting the biometric information from one or more parts of the user, a step of detecting the user's body movement information, a step of reducing the body movement information from the biometric information, and a step of converting the biometric information from which the body movement information is reduced into the character information or the voice information. Thus, it is possible to reduce the body movement information from the biometric information and convert the biometric information into the character information or the voice information.

Further, the conversion unit 106 applies the trained model for the silent speech period and the trained model for the vocalized speech period in accordance with the user's silent speech period and vocalized speech period. Thus, the conversion unit 106 is capable of switching, depending on whether the user is in the silent speech period or the vocalized speech period, from a first conversion method to a second conversion method, or from the second conversion method to the first conversion method, and outputting character information or voice information obtained by conversion by the switched conversion method.

The information processing apparatus 200 can also be provided with a processing unit that evaluates the biometric information detected by the second biometric information detection unit 102 based on a predetermined evaluation standard and deletes character information or voice information corresponding to biometric information that does not satisfy the predetermined evaluation standard.

Thus, according to the present disclosure, it is possible to output the character information or the voice information obtained by conversion with high accuracy.

Further, by converting the character information into the voice information, playback and recording the voice information on an external terminal becomes possible, and the user can listen to played-back audio through earphones. This enables the user to check whether the conversion has been properly performed. Furthermore, the voice information can be transferred via the network and played back and recorded on another external terminal. It is also possible to perform control of an external terminal based on the character information obtained by conversion. By repeatedly executing this series of flows, continuous communication with use of the biometric information becomes possible.

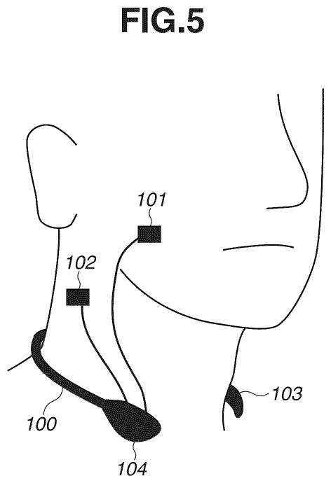

Subsequently, an example of the detection device 100 is illustrated in FIG. 5.

As illustrated in FIG. 5, the user wears the detection device 100 on the neck portion. The detection device 100 is of a neckband-type that is worn so as to sandwich the user's neck portion. The detection device 100 includes the first biometric information detection unit 101, the second biometric information detection unit 102, and the body movement information detection unit 103. The first biometric information detection unit 101, the second biometric information detection unit 102, and the body movement information detection unit 103 are connected to the transmission unit 104 by wires. The transmission unit 104 is capable of transmitting the biometric information detected by the first biometric information detection unit 101, the biometric information detected by the second biometric information detection unit 102, and the body movement information detected by the body movement information detection unit 103 to the outside.

The first biometric information detection unit 101 and the second biometric information detection unit 102 are a three-axis acceleration sensor and a three-axis angular velocity sensor. The three-axis acceleration sensor and the three-axis angular velocity sensor are, sensors capable of measuring, for example, translational accelerations of three axes and angular accelerations of three axes, respectively.

The first biometric information detection unit 101 and the second biometric information detection unit 102 are respectively adhered to the user's cheek and below the user's ear, for example, with a self-adhesive gel. Thus, it is possible to detect the biometric information (acceleration, angular velocity, and the like) with the first biometric information detection unit 101 and the second biometric information detection unit 102. Accordingly, it is possible for each of the first biometric information detection unit 101 and the second biometric information detection unit 102 to detect information regarding the movement of the user's body part.

The body movement information detection unit 103 is a three-axis acceleration sensor and a three-axis angular velocity sensor. The three-axis acceleration sensor and the three-axis angular velocity sensor are, for example, sensors capable of measuring translational accelerations of three axes and sensors capable of measuring angular accelerations of three axes. The body movement information detection unit 103 is installed in the vicinity of the user's neck. The body movement information detection unit 103 is installed at a position that is not affected by the movement of the user's mouth.

The transmission unit 104 transmits the biometric information detected by the first biometric information detection unit 101, the biometric information detected by the second biometric information detection unit 102, and the body movement information detected by the body movement information detection unit 103 to, for example, the Wi-Fi connected information processing apparatus 200. The reception unit 105 of the information processing apparatus 200 receives data. The conversion unit 106 uses the trained model to convert the biometric information from which the body movement information is reduced into the character information or the voice information.

For example, the conversion unit 106 uses sensor information regarding twelve axes in total including three axes×two acceleration sensors and three axes×two angular velocity sensors as input data for a conversion algorithm to convert the biometric information into the character information.

To generate the trained model according to the present embodiment, biometric information in the silent speech period was acquired thirty times from five users with respect to each of twenty sentences. Here, eighty percent of all data was used as training data, and the remaining twenty percent was used as evaluation data. The trained model was created by executing training on a neural network for 500 epochs with a ground truth label obtained by converting a sentence into phonemes with use of training data.

The character information obtained by conversion by the conversion unit 106 is displayed on a display, which is the display unit 107 of the smartphone. An voice information output unit 304 plays back the voice information obtained by conversion by the conversion unit 106.

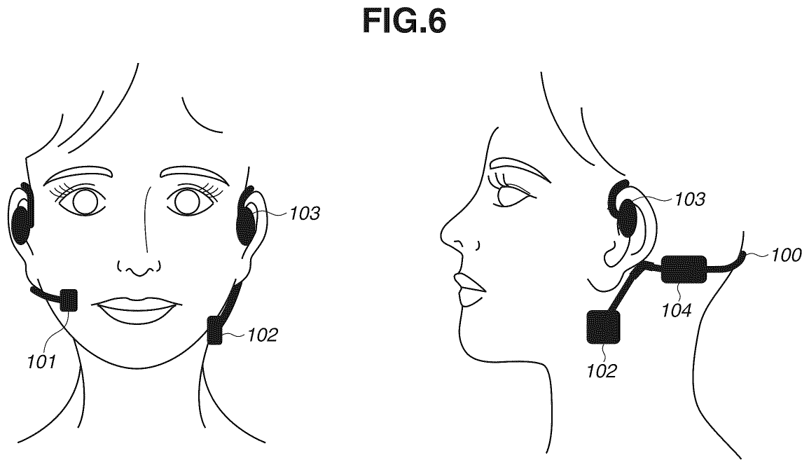

Subsequently, an example of the detection device 100 is illustrated in FIG. 6.

As illustrated in FIG. 6, the user wears the detection device 100 on the neck portion and the ear. The detection device 100 includes a neckband-type portion that is worn so as to sandwich the user's neck portion and an earphone-type portion. The neckband-type portion and the earphone-type portion are connected by a wire. The detection device 100 may include a connecting portion that connects both ends of an elastic member to allow the detection device 100 to be worn on the user's neck portion. The detection device 100 includes the first biometric information detection unit 101, the second biometric information detection unit 102, the body movement information detection unit 103, and the transmission unit 104. The first biometric information detection unit 101, the second biometric information detection unit 102, and the body movement information detection unit 103 are connected to the transmission unit 104. The transmission unit 104 is capable of transmitting the biometric information detected by the first biometric information detection unit 101, the biometric information detected by the second biometric information detection unit 102, and the body movement information detected by the body movement information detection unit 103 to the outside. Furthermore, the transmission unit 104 may have a function of receiving information converted by the conversion unit 106.

Each of the first biometric information detection unit 101 and the second biometric information detection unit 102 is a unit in which the three-axis acceleration sensor, the three-axis angular velocity sensor, and the electromyographic sensor are integrated. The three-axis acceleration sensor and the three-axis angular velocity sensor are sensors capable of measuring, for example, translational accelerations of three axes and angular accelerations of three axes, respectively. A three-electrode Ag electrode is used as an electrode of the electromyographic sensor.

The first biometric information detection unit 101 and the second biometric information detection unit 102 are pressed against the user's cheek and lower jaw portion by a supporting member extending from a sound output unit worn on the user's ears. The first biometric information detection unit 101 and the second biometric information detection unit 102 may be adhered thereto, for example, with a self-adhesive gel to further increase wearability. Hence, the first biometric information detection unit 101 and the second biometric information detection unit 102 are capable of detecting biometric information (various kinds of acceleration and electromyographic potentials). This makes it possible for each of the first biometric information detection unit 101 and the second biometric information detection unit 102 to detect information regarding the movement of the user's body part.

The body movement information detection unit 103 is installed in the vicinity of the user's ear. The body movement information detection unit 103 is installed at a position that is not affected by the movement of the user's mouth.

A computer program that implements the functions of the above embodiments can be supplied to a computer via a network or a memory (not illustrated), and the computer program can be executed by a processor (not illustrated). The computer program is a program for causing the computer to execute the information conversion method described above. In other words, the computer program is a program for implementing functions of an information conversion apparatus by a computer. A memory stores the computer program.

The present disclosure is not limited to the above embodiments, and various changes and modifications can be made without departing from the spirit and scope of the present disclosure. Therefore, the following claims are appended to disclose the scope of the present disclosure.

According to the present disclosure, it is possible to reduce body movement information from biometric information and convert the biometric information into character information or voice information.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. An information conversion system comprising:
a biometric information detection unit configured to detect biometric information from one or more parts of a user;
a body movement information detection unit configured to detect body movement information of the user;
a body movement reduction processing unit configured to reduce the body movement information from the biometric information; and
a conversion unit configured to convert the biometric information from which the body movement information is reduced into character information or voice information.

2. The information conversion system according to claim 1, wherein the biometric information detection unit is an acceleration sensor configured to detect movement of a mouth and a tongue of the user.

3. The information conversion system according to claim 1, wherein the biometric information detection unit is an acceleration sensor and an angular velocity sensor each configured to detect movement of a mouth and a tongue of the user.

4. The information conversion system according to claim 1, wherein the biometric information detection unit is installed in a vicinity of a mouth of the user.

5. The information conversion system according to claim 1, wherein the biometric information detection unit includes a first biometric information detection unit and a second biometric information detection unit, and the first biometric information detection unit and the second biometric information detection unit are installed in different body parts of the user.

6. The information conversion system according to claim 5, wherein the body movement reduction processing unit is configured to subtract a waveform based on the body movement information detected by the biometric information detection unit from a waveform based on biometric information detected by the first biometric information detection unit, and subtract the waveform based on the body movement information detected by the biometric information detection unit from a waveform based on biometric information detected by the second biometric information detection unit.

7. The information conversion system according to claim 6, wherein the conversion unit is configured to use a first waveform obtained by subtracting the waveform based on the body movement information detected by the body movement information detection unit from the waveform based on the biometric information detected by the first biometric information detection unit and a second waveform obtained by subtracting the waveform based on the body movement information detected by the body movement information detection unit from the waveform based on the biometric information detected by the second biometric information detection unit to generate character information or voice information.

8. The information conversion system according to claim 1, wherein the body movement reduction processing unit is configured to reduce the body movement information from the biometric information by subtracting an acceleration detected by the body movement information detection unit from an acceleration detected by the biometric information detection unit or subtracting an angular velocity detected by the body movement information detection unit from an angular velocity detected by the biometric information detection unit.

9. The information conversion system according to claim 1, wherein the conversion unit is configured to use a trained model having been trained by associating biometric information with character information or voice information with use of a correspondence relationship between biometric information and character information or voice information in a plurality of datasets as training data to perform inference on the biometric information from which the body movement information is reduced and output the character information or the voice information.

10. The information conversion system according to claim 1, wherein the body movement information detection unit is an acceleration sensor configured to detect the body movement information of the user.

11. The information conversion system according to claim 1, wherein the body movement information detection unit is an acceleration sensor and an angular velocity sensor each configured to detect the body movement information of the user.

12. The information conversion system according to claim 1, wherein the body movement information detection unit is installed in a vicinity of a neck or an ear of the user.

13. The information conversion system according to claim 1, wherein a sensor used in the body movement information detection unit and a sensor used in the biometric information detection unit are sensors of an identical type.

14. An information conversion method comprising steps of:
detecting biometric information from one or more parts of a user;
detecting body movement information of the user;
reducing the body movement information from the biometric information; and
converting the biometric information from which the body movement information is reduced into character information or voice information.

15. A non-transitory computer-readable storage medium storing a program for causing a computer to execute the information conversion method according to claim 14.
